(19) United States
(12) Patent Application Publication
(10) Pub. No.: US 2025/0361257 A1
(43) Pub. Date: Nov. 27, 2025

(54) SYSTEM AND METHODS FOR SEQUENTIAL DESORPTION OF CANNABIDIOL (CBD) GLYCOSIDE SPECIES

(71) Applicant: TRAIT BIOSCIENCES, INC., LOS ALAMOS, NM (US)

(72) Inventors: Loan HUYNH, LOS ALAMOS, NM (US); Zipeng TIAN, LOS ALAMOS, NM (US); Tyler SMITH, LOS ALAMOS, NM (US); Hanny KANAFANI, LOS ALAMOS, NM (US)

(21) Appl. No.: 18/873,866

(22) PCT Filed: Jun. 9, 2023
(86) PCT No.: PCT/US2023/068177

§ 371 Date: Dec. 11, 2024

Related U.S. Application Data
(60) Provisional application No. 63/351,368, filed on Jun. 11, 2022.
(60) Provisional application No. 63/476,809, filed on Dec. 22, 2022.

(51) Int. Cl. C07H 15/203; C07H 1/06

(52) U.S. Cl. CPC C07H 15/203; C07H 1/06

(57) ABSTRACT

High yield separation and purification of one or more cannabinoid glycoside species from a complex mixture of cannabinoids. In one preferred aspect, the novel systems and methods of the invention can separate one or more cannabinoids and/or cannabinoid glycoside species from a complex mixture, and preferably a quantity of cannabidiol (CBD) glycosides having two UDP-sugar moieties from a complex mixture of CBD and CBD-glycosides having one UDP-sugar moiety.

### CROSS-REFERENCE TO RELATED APPLICATIONS

This International PCT application claims the benefit of and priority to U.S. Provisional Application No. 63/351,368, filed Jun. 11, 2022, and U.S. Provisional Application No. 63/476,809, filed Dec. 22, 2022 the specification, claims and drawings of which are incorporated herein by reference in their entirety.

### TECHNICAL FIELD

The present invention is related to the field of chemical separation and purification, specifically the desorption of specific cannabinoid glycoside species, and preferably select CBD-glycoside species from a complex mixture.

### BACKGROUND

Cannabinoids are a class of specialized compounds synthesized by Cannabis. They are formed by condensation of terpene and phenol precursors. They include these more abundant forms: Δ⁹-tetrahydrocannabinol (THC), cannabidiol (CBD), cannabichromene (CBC), and cannabigerol (CBG). Another cannabinoid, cannabinol (CBN), is formed from THC as a degradation product and can be detected in certain plant strains. Typically, THC, CBD, CBC, and CBG occur together in different ratios in the various plant strains. These cannabinoids are generally lipophilic, nitrogen-free, mostly phenolic compounds and are derived biogenetically from a monoterpene and phenol, the acid cannabinoids from a monoterpene and phenol carboxylic acid and have a C21 base. Cannabinoids also find their corresponding carboxylic acids in plant products. In general, the carboxylic acids have the function of a biosynthetic precursor. For example, the tetrahydrocannabinols Δ⁹- and Δ⁸-THC arise in vivo from the THC carboxylic acids by decarboxylation and likewise, CBD from the associated cannabidiolic acid.

Importantly, cannabinoids are hydrophobic small molecules and, as a result, are highly insoluble. Due to this insolubility, cannabinoids such as THC and CBD may need to be efficiently solubilized to facilitate transport, storage, and adsorption through certain tissues and organs. For example, the metabolism of cannabinoids in the human body goes through the classic two-phases detoxification process of oxidation followed by glucuronidation—which is a form of glycosylation involving the addition of a sugar from UDP-Glucuronic Acid to a cannabinoid. As shown below, the chemical structures of UDP-glucuronic acid and UDP-glucose are similar. As described in, U.S. Pat. No. 8,410,064 by Pandya et al., cannabinoids may be subject to cytochrome P450 oxidation and subsequent UDP-glucuronosyltransferase dependent glucuronidation in the body after consumption. The resulting glucuronide of the oxidized cannabinoids is the main metabolite found in urine, and thus, this solubilization process plays a critical role in the metabolic clearance of cannabinoids.

Moreover, Cannabis has a long history of known medicinal value (1-7). More than 70 cannabinoids with diverse pharmacological properties have been isolated (8). However, known cannabinoids are limited by their hydrophobicity, which curtails some forms of administration and therapeutic use. One available strategy to enhance the solubility of hydrophobic compounds is glycosylation. For example, the generation of water-soluble cannabinoid glycosides has been described by Sayre et al., (See PCT/US18/24409 and PCT/US18/41710, both of which are incorporated herein in their entirety by reference, including examples 1-19, and all specific materials and method) using a fermentation method. Such fermentation is drawing significant attention for natural products due to its capacity to meet industrial demands of water soluble CBD and other cannabinoids. However, strategies for product capture and purification of desired fermentation products remain underdeveloped in the public domain, especially at industrial scale.

As such, it is desirable to chemically separate and isolate one or more cannabinoids and/or cannabinoid glycoside species, for example by the number of sugar moieties from a complex mixture of the same. Use of resins to separate related chemical species has been of great interest for purification because of its simplicity to separate desired and undesired compounds. As described herein, the present inventors provide novel systems and methods of utilizing the solubility and lipophilic properties of CBD-glycoside species to effectively (i) desorb the products from absorbed resins, and (ii) to separate and purify one or more cannabinoid glycoside species from a complex mixture using liquid/liquid extraction and crystallization methods. The present invention can effectively reduce downstream processing cost, enhance purity and yield for target cannabinoid glycoside species, and in particular commercially relevant CBD-glycoside species.

### SUMMARY OF THE INVENTION

One aspect of the present invention includes novel systems, methods, and composition for the separation and purification of one or more cannabinoid glycoside species from a complex mixture. In one preferred aspect, the novel systems and methods of the invention can separate one or more cannabinoids and/or cannabinoid glycoside species from a complex mixture.

Another aspect of the present invention includes novel systems, methods, and composition for the separation and purification of one or more cannabinoid glycoside species from a complex mixture of cannabinoids and cannabinoid glycosides having one or more UDP-sugar moieties (also generally referred to as a “sugar” or “sugar moiety”).

Another aspect of the present invention includes novel systems, methods, and composition for the separation and purification of one or more cannabinoid glycoside species from a liquid complex mixture a complex mixture of cannabinoids and/or cannabinoid glycosides having one or more sugar moieties.

Another aspect of the present invention includes novel systems, methods, and composition for the separation and purification of one or more cannabinoid glycoside species from a resin containing a complex mixture of absorbed cannabinoid glycosides having one or more sugar moieties.

Another aspect of the present invention includes a novel sequential desorption system that simultaneously isolates cannabinoids, and cannabinoid glycosides having one or more sugar moieties during the desorption process, followed by purification. In one preferred aspect, the present invention includes a novel sequential desorption system that simultaneously isolates from a complex mixture, non-glycosylated CBD, CBD having one UDP-sugar moiety (CBD1G), and CBD having two UDP-sugar moieties (CBD2G) during the desorption process, followed by purification of the same.

In another preferred embodiment, the present invention includes novel systems, methods and compositions for the separation and purification of one or more CBD glycoside species from a complex mixture comprising the steps of: 1) Desorption of CBD (which refers generally to a non-glycosylated CBD compound); 2) Desorption of CBD1G and purification, precipitation and/or crystallization; 3) Desorption of CBD2G follow by purification of by crystallization. In this embodiment, the final yield of CBD2G is approximately 85% to 90% at >95% purity.

Additional aspects of the invention may become evident based on the specification and figures presented below.

### BRIEF DESCRIPTION OF THE DRAWINGS

FIG. 1. Chemical structure of (−) trans-cannabidiol-2′-glucoside (CBD1G) and (−)-trans-Cannabidiol-2′,6′-glucoside (CBD2G).

FIG. 2. Desorption and purification chart for CBD1G and CBD2G.

FIG. 3: Percentages of CBD, CBD1G and CBD2G desorbed per sequential desorption.

FIG. 4: Flow chart primary and secondary purification of CBD1G and CBD2G.

FIG. 5. Physical appearance of CBD1G with purities of approximately 41% (left), 70% (middle), and 91% (right).

FIG. 6. Physical appearance of CBD2G with purities of approximately 80% (left), 90% (middle), and 96% (right).

FIG. 7. HPLC-UV chromatogram of CBDGs. The retention time of CBD2G, CBD1G and CBD were 2.9 min, 4.5 min and 6.4 min, respectively.

FIG. 8. Example calibration curves for CBD, CBD1G and CBD2G based on HPLC-UV data (λ=220 nm).

FIG. 9: Alternative schematic flow chart showing additional preferred embodiments of desorption and purification of CBDs species, including CBD, CBDIG, and CBD2G in one embodiment thereof.

FIG. 10: Alternative schematic flow chart showing additional preferred embodiments of desorption and purification of CBDs species from the absorbed PAD900 resin. (1) Desorption of CBD using heptane (HEP), and (2) CBD1G using a mixture of isopropanol (IPA) and heptane, followed by precipitation or crystallization. (3) Desorption of CBD2G using 2-methyl tetrahydrofuran (MTHF) followed by crystallization. Number of cycles of fresh solvents correlate with the amount of the absorbed CBDs species.

FIG. 11: Chemical structure of CBD and exemplary CBD1G and CBD2G.

### DETAILED DESCRIPTION OF THE INVENTION

The purification and isolation of organic compounds is very important to pharmaceutical and food industries. Here, we focus on developing effective methods for extracting and purifying concentrated cannabinoid glycosides (CBDGs) captured from fermentation processes either by using polymer resin or membrane filtration. Resins are capable of selectively capturing the glycosylated cannabinoids produced by fermentation while eliminating or reducing the concentrations of other impurities and cell debris. Alternatively, membrane filtration presents a great advantage to obtain concentrate CBDGs product as the obtained materials is readily for further purification.

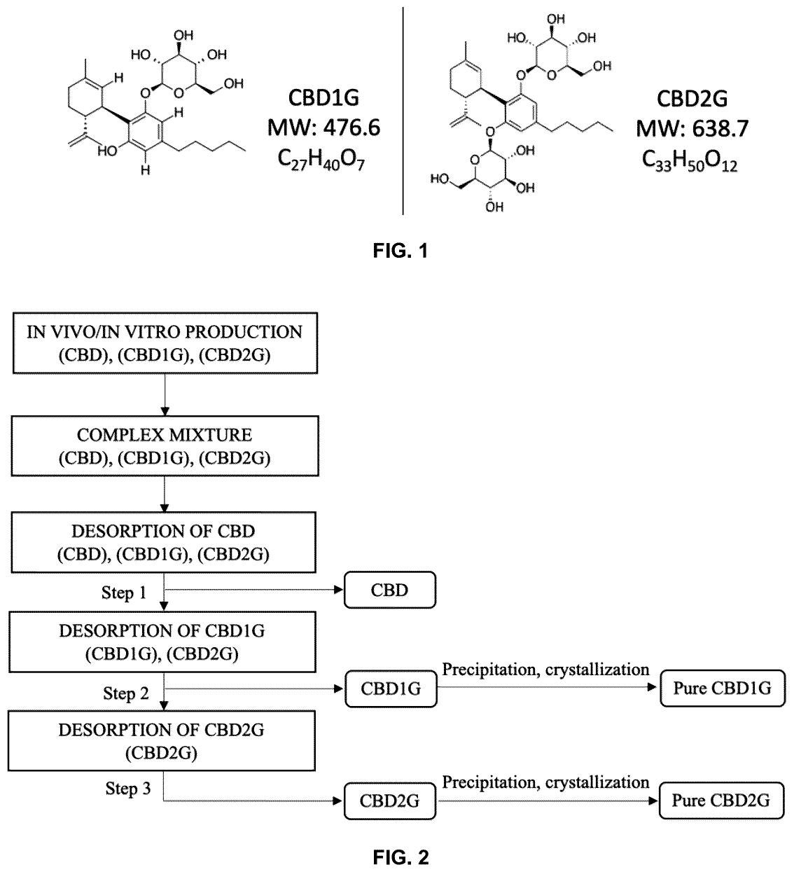

In the present invention, Applicants describe novel desorption methods that sequentially isolate CBD, mono-glycoside CBD (CBD1G) and di-glycoside CBD (CBD2G) (See FIG. 1). These methods utilize the pragmatism that “like dissolves like” to identify suitable solvents for sequentially desorbing CBD, CBD1G and CBD2G compounds from the resin. Several solvent mixtures/ratios were evaluated to determine a sequential procedure by exploring the use of appropriate solvents possessing slightly different polarities. In addition, Applicants also describe herein novel methods for the purification of CBD1G and CBD2G using liquid/liquid extraction, precipitation, and crystallization approaches (See FIG. 2). Using these methods, the overall purity of CBD1G and CBD2G is approximately 95%-97%.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

In one preferred embodiment, the present inventors described novel methods for the purification of glycosylated cannabinoid derivatives, including CBD1G and CBD2G, sometimes referred to a CBD1XGly and CBD2XGly, respectively. These methods involve novel applications of filtration and crystallization approaches. Modulation of solvent mixtures and other procedural details allows the preferential enrichment, and therefore separation, of individual cannabinoids and/or derivative compounds. Resulting single-final-product with the highest purity of 96% or higher. Total yields ranged from 60-90%. Solvent recycling and multi-crop crystallization strategies were used to increase efficiency.

In the preferred embodiment described below, organic solvents, such as 2-methyl tetrahydrofuran was used for the desorption of CBD2G. The extracted solution was evaluated with ethyl acetate, isopropyl acetate being used to extract CBD and CBDGs from the fermentation broth containing a complex mixture as described herein using liquid/liquid extraction. Alternatively, PuroSorb™ PAD900 resin was used to capture CBD and CBDGs from the fermentation broth containing a complex mixture, followed by sequential desorption at room temperature or using a Soxhlet extractor.

### Example 1: Sequential Desorption Procedure Including Liquid/Liquid Extraction, Precipitation, and Crystallization

### 1.1. Extraction of CBDGs Liquid/Liquid Extraction Method

Referring generally to FIG. 2, in one embodiment the invention includes can include establishing a complex mixture of cannabinoids, and preferably a complex mixture of CBD and CBDGs. Embodiments for the in vitro production of CBDGs is described by Zipp et al., PCT/US2016/05312, while embodiments for the in vivo production of CBDGs in plant and yeast-based systems is described by Sayre et al., PCT/US18/24409 and PCT/US18/41710, all of which are incorporated herein by reference. These in vitro and in vivo methods for producing cannabinoid glycosides are incorporated specifically herein reference. Moreover exemplary glycosyltransferases (UGTs) enzymes having activity toward one or more cannabinoids, and in particular CBD is described by Sayre et al., in PCT/US18/24409, PCT/US18/41710, and PCT/US21/20040. These UGT sequences are incorporated specifically herein by reference. The above references describe methods for the in vitro and in vivo production of a complex mixture of CBD and CBDGs, and in particular CBD, CBD1G, and CBD2G respectively, such methods being specifically incorporated herein in their entirety.

Concentrated aqueous solutions of CBDGs obtained from fermentation, generally referred to as a complex mixture, were extracted with 2-methyl tetrahydrofuran was used for the desorption of CBD2G. The extracted solution was evaluated. Alternatively, ethyl acetate or isopropyl acetate were also evaluated for the extraction of CBDGs. Solutions of CBDGs were washed with aqueous solutions of saturated sodium bicarbonate and sodium chloride. The organic layer was collected and concentrated under vacuum using a rotary evaporation to obtain off-white solid CBD2G which was further purified as outlined in the purification section.

1.2. Sequential Desorption of CBDGs from Resin at Room Temperature

Dry resin was weighed and transferred to a stainless-steel container with a 200 μm mesh for sequential desorption for the extraction of CBDGs. This entire apparatus was held by a glass beaker. Three sequential steps were used for the desorption of CBD and CBDGs: (i) desorption of CBD; (ii) desorption of CBD1G and (iii) desorption of CBD2G.

### (i) Desorption of CBD.

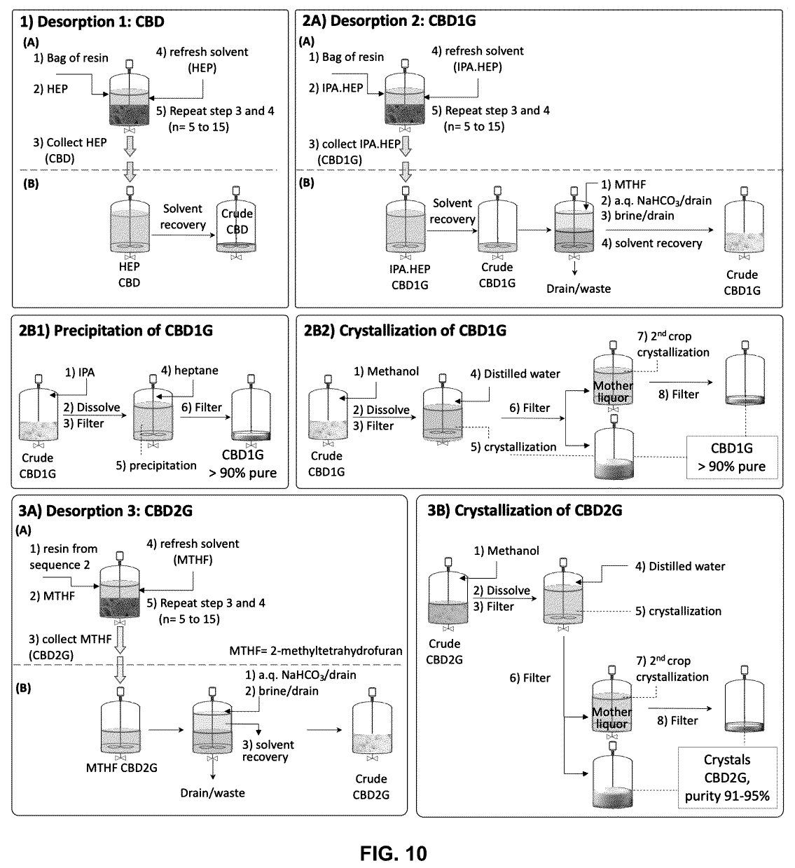

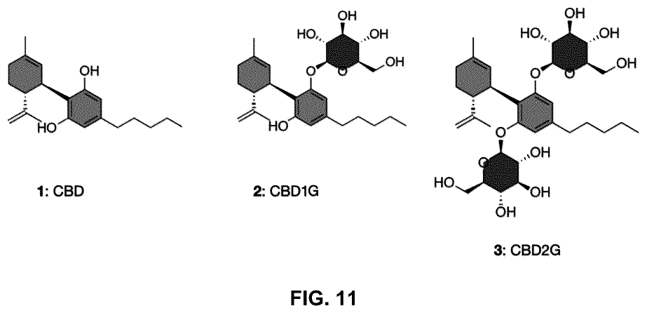

As described as step 1 in FIGS. 2 and 10, desorption CBD was carried out using heptane. Other hydrophobic solvents could be utilized in place of n-heptane, e.g., n-hexane, or pentane. Specifically, 6 mL of heptane was added per gram of dried resin and incubated for 60 min with occasional stirring, followed by draining to collect the solution of heptane containing CBD. This step was repeated for 15 to 20 cycles or until ˜90% of CBD was desorbed, using clean solvent for each cycle. Upon completion, used heptane was recycled by rotary evaporation and subsequent condensation.

### (ii) Desorption of CBDIG.

As described as step 2 in FIGS. 2 and 10, using the same procedures for the desorption of CBD, a binary mixture of isopropanol (IPA) and n-hexane (HEX) was used at a ratio of 1:9, v/v (IPA/HEX) for the desorption of CBDIG. The extracted solution was evaluated using HPLC-UV (Table 1). Other polar solvents could be utilized in place of isopropanol, (e.g., acetone or alcohols) with the ratio in the range of 3% to 20% in the binary mixture. Alternatively, steps (i) and (ii) could be combined to desorb CBD1G and CBD using a mixture of isopropanol and n-heptane. Combined solutions of CBD1G were concentrated to obtain dry solid CBD1G which was then dissolved in 2-methyl tetrahydrofuran and washed with aqueous solutions of saturated sodium bicarbonate and then sodium chloride. The organic solvent layer was then recovered and dried by rotary evaporation to obtain semi-clean CBD1G.

(iii) Desorption of CBD2G.

As described as step 3 in FIGS. 2 and 10, using similar procedures for the desorption of CBD or CBD1G, 2-methyl tetrahydrofuran was used for the desorption of CBD2G. The extracted solution was evaluated using HPLC-UV (Table 2). Alternatively, other polar solvents were also evaluated including ethyl acetate, isopropyl acetate, methanol, ethanol, and isopropanol. Combined solutions of 2-methyl tetrahydrofuran containing CBD2G were washed with aqueous solutions of saturated sodium bicarbonate and sodium chloride. The organic layer was collected and concentrated under vacuum using rotary evaporation to obtain off-white solid semi-clean CBD2G, which was further purified as outline in the purification section.

1.3. Sequential Desorption of CBDGs from Resin Using Soxhlet. (Alternative Step 1)

In one preferred embodiment, dry resin was weighed and transferred to a stainless-steel container with a 200 μm mesh that was used as a thimble for the Soxhlet extractor.

### (i) Desorption of CBD and CBD1G.

As described as steps 1-2 in FIG. 2, similar to the methods used for sequential desorption at room temperature, hexane or heptane was used to desorb CBD at elevated temperature. Following the desorption of CBD, a mixture of propanol and hexane (3.6:94.6, v/v) was used to desorb CBDIG. The extraction was conducted at 50° C. for ˜50 cycles of extraction using a Soxhlet extractor. During this extraction, CBD2G slowly precipitated out of solution. An off-white powder of CBD1G with a purity of ˜90% was obtained and dried for further purification.

### (ii) Desorption of CBD2G.

As described as steps 1-2 in FIG. 2, subsequently, a binary mixture of propanol and hexane (19:81, v/v) was used to desorb CBD2G. The extraction was conducted at 50° C. for ˜50 cycles of extraction using a Soxhlet extractor. Alternatively, a binary mixture of acetone and hexane at a ratio of 45:55 (v/v) was also used to extract CBD2G. During this extraction, CBD2G slowly precipitated out of solution. A light-yellow powder of CBD2G with a purity of ˜85% was obtained and dried for further purification.

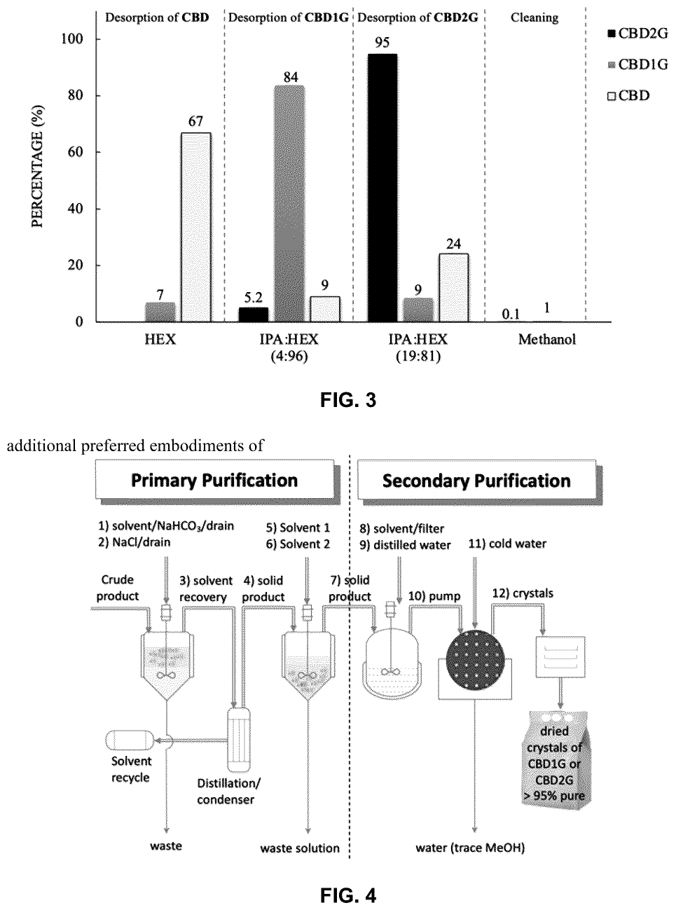

The amount and percentage of CBDGs per sequential desorption was evaluated by HPLC-UV analysis. Using the Soxhlet extraction method, the desorption efficiency is 98% to 99%. Raw CBDGs products were well separated. The products obtained from desorption of CBD1G sequence contain 94% CBD1G and ˜4.5% CBD2G. The products obtained from desorption of CBD2G sequence contain 88% CBD2G and ˜10% CBD1G (FIG. 3).

### 1.4 Purification

Purification of CBDGs was carried out in 2 steps as shown in FIG. 4.

### 1.4.1 Purification of CBD1G Post Desorption/Extraction by Precipitation and Crystallization.

Purification of CBD1G in this embodiment was carried out in 2 steps:

### (i) Primary Purification of CBD1G by Precipitation or Crystallization.

Crude CBD1G obtained from liquid/liquid extraction was added to acetone at a concentration of 60 to 80 mg/ml (CBD1G/acetone) and stirred for 30 to 60 min at ˜40° C. followed by hot filtration. Next, heptane was added to obtain a solution of acetone: heptane in a ratio of 1:3 (v/v), which was stored at room temperature overnight to allow impurities to precipitate out of solution. The clarified solution was decanted into a separate glass container and covered with aluminum foil. Following storage at room temperature for a minimum of 3 days, needle-shaped white crystals formed. After collecting and drying these crystals, they were resuspended in methanol and analyzed by HPLC-UV, which revealed that their major constituent was CBG1G, and that the purity was ˜70% to 85%.

Separately, various ratios of acetone (or propanol) and heptane (or hexane), were also evaluated. Crystals of CBD1G with similar morphologies and purities were also obtained in these solvents, but the crystallization rate was slower.

### (ii) Secondary Purification of CBD1G by Crystallization.

The dried CBD1G obtained from the primary purification was further purified by a secondary crystallization using acetone and deionized water. The dry powder of CBD1G was dissolved in acetone at a concentration of 30-45 mg/mL (CBD1G/acetone) at ˜50° C., followed by filtration using 0.2 mm PVDF filter. Distilled or deionized water was then added to the permeant to obtain a solution containing 40 to 50% water, which was stored at room temperature for 24 to 48 hours to obtain white needle-like crystal of CBD1G. The liquid was removed using vacuum filtration, followed by washing with minimum volume of ice-cold distilled or deionized water.

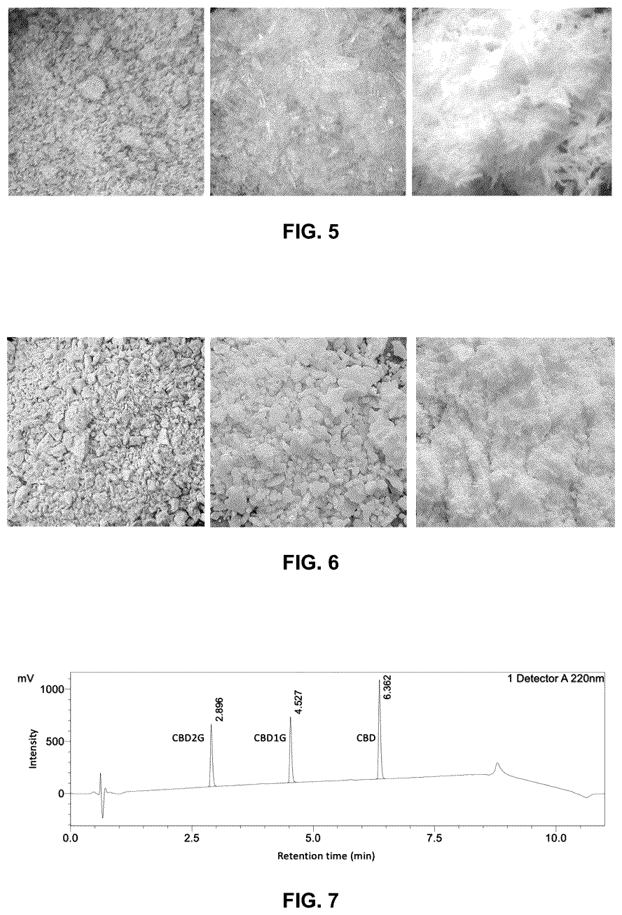

Using similar protocols, methanol was used in place of acetone. Solutions of CBD1G at 140 to 160 mg/mL were filtered using 0.2 mm PVDF filter. Then, distilled or deionized water was added to the permeant to obtain a solution of methanol: water at a ratio of 3:2 (v/v). This solution was stored at room temperature overnight to obtain white needle like crystal of CBDIG. The obtained CBD1G crystals were dried in a vacuum oven for 24 hours at 60° C. to obtain dried CBD1G with purity of 88±3%. The total yield was 60±20%. Shown in FIG. 5 is the physical appearance of CBD1G at various purities.

### 1.4.2 Purification of CBD2G Post Desorption/Extraction by Precipitation and Crystallization

Purification of CBD2G was carried out in 2 steps.

### (i) Primary Purification of CBD2G by Precipitation or Crystallization.

Crude CBD2G was added to acetone at a concentration of 80 mg/ml (CBD2G/acetone) and stirred for 30 to 60 min at ˜40° C., followed by filtration. This solution was covered with aluminum foil and stored at room temperature to collect the first crop of CBD2G crystals. The purity of these crystals was 82% to 86%. To improve total yield, the remaining solution (with uncrystallized CBD2G and other impurities) was used to generate a second crop of crystals by adding heptane to the solution at a concentration of 25 to 75% (v/v). This solution was covered with aluminum foil and stored at room temperature for 24 to 48 hours to allow precipitation or crystallization of CBD2G. The obtained CBD2G was then dried in a vacuum oven for 8 to 24 hours at 90-100° C. to obtain CBD2G with 89±4% purity. Combining both crops of crystals, the total yield was 90±2%.

Alternatively, isopropanol was evaluated in replacement of acetone, whereas other hydrophobic solvents (e.g., hexane or pentane) could be used in place of heptane. In cases where isopropanol (3 to 10%) and heptane were used, an amorphous solid precipitate containing CBD2G was obtained.

### (ii) Secondary Purification of CBD2G by Crystallization.

The obtained dried CBD2G of 86% purity was further purified by a secondary crystallization using methanol and distilled water. The dry powder of CBD2G was dissolved in methanol at a concentration of 140 mg/mL up to 180 mg/mL at ˜40° C., followed by filtration using a 0.2 mm PVDF filter. Distilled or deionized water was then added to the solution of CBD2G in methanol to obtain a solution containing 40 to 45% water, which was filtered again using a 0.2 mm PVDF filter. Crystallization was observed a few minutes after the addition of water. The final solution was stored at room temperature for 24 hours to obtain white needle-like crystal of CBD2G. The liquid was removed using vacuum filtration and saved, followed by washing with minimum volumes of ice-cold distilled or deionized water. The liquid removed from the crystals was combined with the liquid from washing the crystals and filtered to remove impurities followed by an additional 48-hour recrystallization to collect a second crop of CBD2G.

Alternatively, acetone or ethanol was also evaluated in replacement of methanol for the crystallization of CBD2G. Similarly, crystals of CBD2G were obtained when acetone was used, while a white amorphous solid precipitate was obtained when ethanol was used. The obtained CBD2G crystal was dried in a vacuum oven for 24 hours at 80-100° C. to obtain CBD2G at 96±1% purity. Combining both crops of crystals, the total yield was 73±17%. Shown in FIG. 6 is the physical appearance of CBD2G at various purities.

### Example 2: Materials and Methods

### Materials.

PuroSorb™ PAD900 resin was purchased from Purolite (Purolite, USA). Clean and dried resin containing approximately 15% of CBD and CBDGs (% wt.) was used in this study. As an example, the amount of CBD and CBDGs absorbed to 1 gram of dried resin was 0.074 g CBD2G, 0.087 g of CBD1G and 0.013 g of CBD.

### Chemicals and Reagents.

Sodium bicarbonate, sodium chloride, formic acid (99%) and ammonium formate (97%) were purchased from Oakwood Chemicals (Oakwood Products, Inc., SC, USA). Ethanol, methanol, n-propanol, isopropanol, ethyl acetate, dichloromethane, methanol, n-heptane, heptane, n-hexane, 2-methyltetrahydrofuran, ethyl acetate, isopropanol acetate HPLC grade acetonitrile and HPLC grade deionized water were purchased from Oakwood Chemicals (Oakwood Products, Inc., SC, USA).

### High-Performance Liquid Chromatography-Ultraviolet (HPLC-UV).

HPLC-UV analyses were carried out on a Shimadzu 2050c LC equipped with a split/splitless injector and an autosampler. Separations were carried out on a Raptor C18 column (100 mm length×4.6 mm inner diameter, 5 μm particle size). The injection volume was 5 μL. Mobile phases were (A) water with 5 mM ammonium formate and 0.1% formic acid—and (B) acetonitrile containing 0.1% formic acid under the following gradient program: 0.1 min, 92% A and 8% B, 8.0 min, 0% A, 100% B; at 8.1 min, 92% A and 8% B; at 11.0 min, 92% A and 8% B. The flow rate was set at 1.5 mL/min from 0 to 8.1 min; and 2.5 mL/min from 8.11 min to 11 min, with oven temperature of 40° C. CBD2G, CBD1G and CBD were detected at 220 nm, at retention times of 2.9 min, 4.5 min and 6.4 min, respectively (FIG. 7).

### HPLC-UV Calibration.

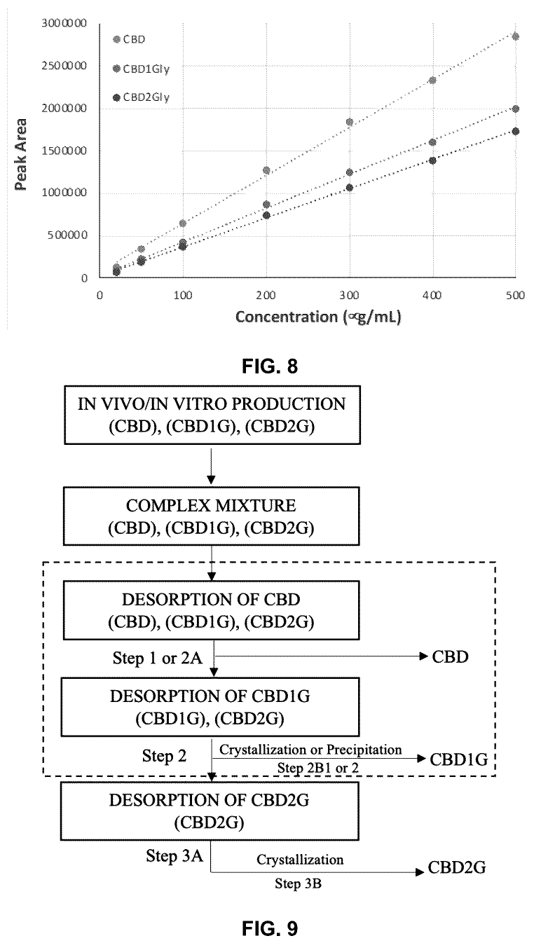

Calibration curves were prepared with CBD2G, CBDIG, and CBD ranging from 20 μg/mL to 500 μg/mL in HPLC-grade methanol. Seven (7) calibration standards were used to create a linear correlation for quantitative evaluation of each compound of interest. The linear correlation coefficient for each compound was R2≥0.997 (FIG. 8).

### Example 3. Alternative Sequential Desorption Procedure

As outlined in FIG. 9, the sequential desorption procedure of the invention is divided into four steps: (1) Desorption of CBD: (2) Desorption of CBD1G by purification by precipitation or crystallization; (3) Desorption of CBD2G follow and purification of by crystallization. The process of the invention has demonstrated a final yield of approximately 85% to 90% at >90% purity for CBD2G. The amount of CBDs species absorbed on the 21 gram of oven dry resin is summarized in Table 3.

### Production of Complex CBD Mixture

Referring generally to FIG. 9, in one embodiment the invention includes establishing a complex mixture of cannabinoids, and preferably a complex mixture of CBD and CBD-glycosides. Embodiments for the in vitro production of cannabinoid glycosides is described by Zipp et al., PCT/US2016/05312, while embodiments for the in vivo production of cannabinoid glycosides in plant and yeast-based systems is described by Sayre et al., PCT/US18/24409 and PCT/US18/41710. These in vitro and in vivo methods for producing cannabinoid glycosides are incorporated specifically herein reference. Moreover exemplary glycosyltransferases (UGTs) enzymes having activity toward one or more cannabinoids, and in particular CBD is described by Sayre et al., in PCT/US18/24409, PCT/US18/41710, and PCT/US21/20040. These UGT sequences are incorporated specifically herein by reference.

The above references describe methods for the in vitro and in vivo production of a complex mixture of CBD and CBD-glycosides, and in particular CBD, CBD1G, and CBD2G respectively, such methods being specifically incorporated herein in their entirety.

### Desorption of CBD

As shown in Step 1A of FIG. 10, the process of separating cannabinoid species from a complex mixture is initiated with the desorption of CBD using a hydrophobic or non-polar solvents, such as heptane, n-heptane, n-hexane, or pentane, or other hydrophobic solvents known in the art. In this embodiment, a resin, in this embodiment being a Purolite™ PAD900 resin, containing a complex mixture of CBD, CBD1G and CBD2G is positioned within a container containing the solvent. The solvent can be refreshed, and this step repeated for 15 cycles in order to allow CBD to desorb from the resin.

As shown in Step 1B of FIG. 10, the solution obtained from cycles 1 to 15 is combined and the solvent recovered forming a crude CBD mixture that can be dried down for further purification of CBD1G and CBD2G.

### Desorption of CBD1G and CBD

As shown in FIG. 10, steps 1 and 2 for the desorption of CBD and CBD1G can be combined to desorb CBD1G and CBD in a single step using a solvent mixture, and in this preferred embodiment, a binary mixture of isopropanol and n-heptane, follow by isolation and purification of CBD1G. As shown in Step 2A, as binary mixture of isopropanol and heptane, (which may be substituted with, for example, n-hexane) was used at a ratio of 1:9, v/v (IPA: HEP). Other polar solvents could be used in replacement of isopropanol, include, for example acetone, with the ratio in a range of 3% to 20% in the binary mixture. Again, as shown in Step 2A, this step was repeated for 15 cycles to allow all CBD1G and CBD to desorb from the resin. The solution obtained from cycles 1 to 15 was combined and dried down for further isolation and purification of CBD1G.

Again referring to Step 2A, parts (A) and (B), the combined desorption of CBD and CBD1G from the resin was accomplished using a binary mixture of isopropanol (IPA) and heptane (at a ratio of 1:9, v/v (IPA: HEP) using the following sequential steps:
(1) First, the dry resin containing a complex mixture of CBD, CBD1G and as described later CBD2G, was transferred to a stainless steel container with a 200 mesh filter.
(2) Next a mixture of IPA: HEP (1:9, v/v) was added to the resin at a ratio of 3.34:1 (v/w, L/Kg).
(3) Another mixture of IPA: HEP (1:9, v/v) was added to the resin at a ratio of 6.2:1 (v/w, L/Kg).
(4) The resin was incubated with occasional mixing for 0.5 hour to allow desorption of CBD1G and CBD.
(5) The mixture of IPA: HEP containing CBD1G and CBD was then collected.
(6) Steps 3 to 5 may be repeated, up to optimally 15 or more cycles or until the CBD1G and CBD has been fully desorbed from the resin.
(7) The solvent the solution containing CBD1G and CBD was recovered, with the dried product stored for further isolation and purification of CBDIG.

The obtained solutions per cycle was evaluated by HPLC-UV. Overall, a total amount of 1.817 g CBD1G and 0.263 CBD was obtained from this step. The extraction efficiency is 99.6% for CBD1G and CBD. Detail of the amount of desorb per cycle is provided in Table 4.

The resin, having had the CBD species desorbed, now contains a complex mixture of CBD1G and CBD2G. As shown in step 2A (B) (FIG. 10), after finishing the desorption of CBD and CBD1G, the crude composition can be purified to isolate the CBD1G from the solution. In this embodiment, the crude CBD1G can be dissolved in 2-methyl tetrahydrofuran (MTHF) and washed with 10% of aqueous solution of NaHCO₃ at a ratio of 1:1 (v/v). After 24 hours of stirring at room temperature, the aqueous layer of NaHCO₃ may be removed. The organic layer may then be washed with equivalent volume of brine for 24 hours. Finally, the organic layer was collected and concentrated under vacuum using a rotated evaporation to obtain off white solid CBD1G.

### Purification of CBD1G Via Precipitation

As shown in FIG. 10, step 2B1 includes the purification of CBD1G by precipitation. In this embodiment, crude CBD1G and CBD obtained from step 2 is further purified by precipitation and crystallization. Initially, crude product of CBD1G and CBD was dissolved in IPA at a concentration of ˜50 mg/ml., followed by addition of HEP to reach a ratio of 1:9 (IPA: HEP, v/v). Solution was stirred at room temperature for 48 hours to obtain white precipitation of CBD1G at ˜75% purity. Further purification was executed using a mixture of IPA.HEP at a ratio of 3:97 (v/v).

### Purification of CBD1G Via Crystallization

Referring again to FIG. 10, step 2B2 shows the alternative and/or additive step of purifying CBD1G by crystallizing. In case CBD was desorb prior to the desorption of CBDIG, crude product obtained from step 2 can be further purify by crystallization. Alternatively, in one embodiment CBD is removed by precipitation step as outline above to obtained crude CBD1Gly. In this embodiment, the obtained dry crude CBD1G was dissolved in methanol at a concentration of 100 mg/mL, followed by filtration. An optional equivalent volume of distilled water was then added to the solution of CBD1G in methanol (or acetone) to obtain a solution of methanol (or acetone) and water at a final concentration of 50 mg/mL. The solution was then filtered again prior to allowing crystallization. The filter the solution was then exposed to open air and allowed to slowly evaporate at room temperature for 24 to 48 hours, until the total solution volume was approximately reduced by half. White needle-like crystal was obtained in a yellow solution. The liquid was gently removed using funnel with filter paper to collect white crystals which was washed with minimum volume of ice-cold distilled water and dried in the vacuum oven for 24 hours to obtained CBD1G with >95% purity.

### Desorption of CBD2G

Referring again to FIG. 10, step 3A shows the desorption of CBD2G from the resin. In this embodiment, after finishing the desorption of CBD1G and CBD, the container of resin from sequence 2 was contacted with 2-methyl tetrahydrofuran (MTHF) to collect CBD2G. This step was repeated for 15 cycles to allow most of the CBD2G to desorb from the resin. Combining solution obtained from cycles 1 to 15 was washed with 10% of aqueous solution of NaHCO₃ at a ratio of 1:1 (v/v). After 24 hours of stirring at room temperature, the aqueous layer of NaHCO₃ was removed. The organic layer was then washed with equivalent volume of brine for 24 hours. Finally, the organic layer was collected and concentrated under vacuum using a rotated evaporation to obtain off white solid CBD2G.

In a preferred embodiment, desorption CBD2G using MTHF as shown in FIG. 10, Step 3A can include the following sequential steps:
(1) To the resin obtained from step 1, add MTHF to the resin at a ratio of MTHF:resin, 3.1:1 (v/w, L/Kg).
(2) Incubate the resin with occasionally mixing for 0.5 hour to allow sufficient desorption of CBD2G.
(3) Collect the MTHF solution containing CBD2G.
(4) Optionally repeat steps 1 to 3 up to 15 or more cycles.
(5) To the combined solution of MTHF, add an equivalent volume of NaHCO₃ solution (˜8 to 10%). Stir overnight, then remove the aqueous solution.
(6) To the solution of MTHF, add an equivalent volume of brine. Stir at RT or 60° C. overnight, then remove the brine solution.
(7) Recover the MTHF solvent to collect off white dried solid of CBD2G.

The obtained solutions per cycle was evaluated by HPLC-UV. Overall, a total amount of 1.55 g CBD2G was obtained from this step. The extraction efficiency is 96% for CBD2G. Detail of the amount of desorb per cycle is provided in Table 5.

### Purification of CBD2G Via Crystallization

Referring again to FIG. 10, in step 3B the crude CBD2G obtained from step 3A is further purified by crystallization. In this embodiment, the obtained dry crude CBD2G was dissolved in methanol at a concentration of 100 mg/mL, followed by filtration. An equivalent volume of distilled water was then added to the solution of CBD2G in methanol to obtain a solution of methanol and water at a final concentration of 50 mg/mL. The solution was then filtered again prior to allowing crystallization. The filter the solution was then exposed to open air and allowed to slowly evaporate at room temperature for 24 to 48 hours, until the total solution volume was approximately reduced by half. White needle-like crystal was obtained in a yellow solution. The liquid was gently removed using funnel with filter paper to collect white crystals which was washed with minimum volume of ice-cold distilled water and dried in the vacuum oven for 24 hours to yield 85% of CBD2G (based on the amount of CBD2G in the raw materials that was used for crystallization) with >90% purity. The washed-water with the yellow mother liquid was combined and filtered to further remove impurity followed by an additional 48 hours recrystallization to collect an additional ˜10 to 15% of CBD2G.

In an alternative embodiment, a mixture of IPA: HEP (3:97, v/v) may be used to wash the crude CBD2G to remove CBD1G and/or CBD, and other impurities prior to crystallization step.

### Materials and Exemplary Yield

In the above embodiment the following materials were used to obtain an isolated CBDIG or CBD2G: Isopropanol, n-heptane, methanol, aqueous solution of 8% to 10% (w %) NaHCO₃, and aqueous solution of 10% (w %) NaCl are used. An amount of 21 grams of dried resin was used for this study. The amount of CBDs species absorbed on the 21 gram of oven dry resin is summarized in Table 3.

### Example 4. Alternative Sequential Desorption Procedure Using Soxhlet Extraction

### Soxhlet Solid-Phase Extraction

In an alternative embodiment, the current invention includes novel systems and method to sequentially desorb CBD, CBD1G and CBD2G in separate fractions under a close system and high temperature. In the this preferred embodiment, the sequential desorption includes is a Soxhlet solid-phase extraction apparatus. As further described below, the inventive method can be scaled using an extractor machine that is equipped with an extraction tank, solvent concentration, condenser and pump. The obtained CBD1G and CBD2G can undergo one or more purification step prior to crystallization to obtain pure products. By selecting suitable solvents, sequential desorption methods using Soxhlet apparatus allows efficient desorption with less solvents, and produce higher purity CBD1G and CBD2G. This method is further applicable to various resin-containing CBDs including PAD900, silica gel and other types of resins as generally described herein.

### Sequential Desorption CBD Species Soxhlet Solid-Phase Extraction

As described below, in one embodiment, an exemplary amount of 5.8 of PAD900 (unconditioned) containing 637 mg CBDs (i.e. 0.103 g of CBD per gram of resin, and separately 5.8 g PAD900 (unconditioned) containing 976 mg CBDs (0.164 g of CBD per gram of resin) was used. Desorption was carried out using a 500 mL and 1000 mL capacity Soxhlet apparatus, having dried resin loaded into the Soxhlet using baskets to create solvent-access-surface area.

In a first sequence, desorption of CBD and CBD1G species can be carried out using the Soxhlet apparatus. In this embodiment, a resin having a complex mixture containing CBD, CBD1G, and CBD2G is established. Desorption of CBD is initially carried out by application of a non-polar solvent, and preferably hexane to the complex mixture. In this embodiment, one or more resins are rinsed with hexane, preferably for multiple cycles which may range between 2-30 or more cycles, using Soxhlet extractor to circulate the solvent. Notably, this first sequence is optional, as it can be bypassed and go straight to sequence 2. As noted above, in this first sequence both CBD1G, and CBD are co-desorbed as a mixture.

In a second sequence, desorption of CBD1G species can be carried out using the Soxhlet apparatus. In this embodiment, a resin having a complex mixture containing a quantity of CBD1G is established. Desorption of CBD1G is initially carried out by application of a non-polar solvent, and preferably hexane as well as a polar solvent, such as isopropanol, to the complex mixture. In a preferred embodiment, desorption of CBD1G is carried out by application to the resin a quantity of hexane containing approximately between 3.6 to 4% by volume of isopropanol. In this embodiment, one or more resins are rinsed with a mixture of isopropanol and hexane, preferably for multiple cycles which may range between 2-30 or more cycles, using Soxhlet extractor to circulate the solvent as shown in FIG. 9 generally.

In a third sequence, desorption of CBD2G species can be carried out using the Soxhlet apparatus. In this embodiment, a resin having a complex mixture containing a quantity of CBD2G is established. In a preferred embodiment, desorption of CBD2G is carried out by application to the resin a quantity of hexane containing approximately 19% by volume of isopropanol. In this embodiment, one or more resins are rinsed with a mixture of isopropanol and hexane, preferably for multiple cycles which may range between 2-30 or more cycles, using Soxhlet extractor to circulate the solvent as shown in FIG. 9 generally.

The amount and percentage of CBD and CBDGs species per sequential desorption as described below was evaluated by HPLC-UV analysis, showing the desorption efficiency of between 96% to 99%. The exemplary sequences described above yielded the following:

Exemplary Sequence 1: Using a single solvent hexane, a mixture containing 30% CBD1G and 70% of CBD was obtained, calculated based on the total amount of CBD and CBDGs obtained from Sequence 1.

Exemplary Sequence 2: Using isopropanol: hexane (4:96, v/v), a mixture containing 4.5% CBD2G, 94% CBD1G and 1.5% of CBD1G-OH and CBD was obtained, calculated based on the total amount of CBD and CBDGs obtained from sequence 2.

Exemplary Sequence 3: Using isopropanol: hexane (19:81, v/v), a mixture containing 88% CBD2G, 11% CBD1G and 1% of CBD1G-OH and CBD was obtained, calculated based on the total amount of CBD and CBDGs obtained from sequence 3.

As noted elsewhere, other non-polar solvents such as heptane, pentane, cyclohexane and their isomers can be used in replacement of hexane. Additionally, alternative polar solvents (e.g. n-propanol, acetone) that are miscible with the non-polar solvent and able to form an azeotrope mixture with the non-polar solvent could be used in replacement of isopropanol.

### Purification of CBDGs Desorbed Using Species Soxhlet Solid-Phase Extraction

The invention further provides methods of purifying CBDGs from the extracts obtained from sequences 1, 2 and/or 3 described above. In one embodiment, the invention includes the purification of CBD1G. In this embodiment, the crude CBD1G, obtained from sequence 2 (see above), is dried and washed with one or more non-polar solvents, such as heptane, hexane and/or their isomers, optionally with a small volume (1 to 10%) of polar solvent such as isopropanol, or acetone to remove CBD species and other impurities. The obtained solid is then dried and further purified by a two steps precipitation/crystallization as outlined below:

First, the crude product of CBD1G is dissolved in a polar solvent, such as isopropanol or acetone at a concentration of ˜25 to 50 mg/mL. The resulting solution of CBD1G is filtered followed by addition of a non-polar solvent, such as heptane to create a mixture with 75% to 95% of heptane. The precipitated material that occurs within 1 hour is removed. Then, the solution is open to air at room temperature for 48 to 72 hours to reduce the volume of solvent by about 25% to 30% to obtain white precipitate/crystals of CBD1G with approximately 75%-90% purity.

Second, the product of CBD1G from step 1 above is dried and then dissolved in a polar solvent, such as methanol, ethanol and/or acetone, at a concentration of approximately 40 to 60 mg/mL, followed by filtration. An equivalent volume of distilled water is then added to the solution of CBD1G in the polar solvent, such as methanol. The solution is open to air at room temperature for 48 to 72 hours to reduce the volume of solvent by approximately 40% to 60% to obtain white needle-like crystal of CBDIG. The liquid is gently removed using a funnel with filter paper to collect the crystals which is quickly rinsed with a minimum volume of ice-cold distilled water and dried in the vacuum oven for 24 hours to obtain CBD1G with approximately >95% pure.

The invention further includes the purification of CBD2G. In this embodiment, crude CBD2G obtained from sequence 3 described above is dried and is further purified by a two steps precipitation/crystallization as outlined below:

First, crude CBD2G is dissolved in a polar solvent, such as acetone at a concentration of 50 to 100 mg/mL. Next, the solution is heated to approximately 50° C. to enhance the solubility of CBD2G in the solvent, in this case being acetone. Alternatively, a solution of CBD2G in a polar solvent, such as acetone could be prepared at room temperature at lower concentration of CBD2G, such as 30 to 40 mg/mL. After filtration of the CBD2G solution, a non-polar solvent, such as heptane is added to create a solution of a non-polar solvent containing 25% to 75% of acetone by volume. The solution is open to air at room temperature for 48 to 72 hours to reduce the volume of solvent by approximately 25% to 40% to collect off white precipitation or crystallization of CBD2G with approximately >95% pure.

Second, crude CBD2G obtained from step 1 is dissolved in a polar solvent, such as methanol, ethanol or acetone, at a concentration of 100 to 120 mg/mL, followed by filtration. An equivalent volume of distilled water is then added to the solution of CBD2G in the polar solvent, in this example being methanol. Then, the solution is open to air at room temperature for 48 to 72 hours to reduce the volume of solvent by approximately 40% to 50% to obtain white needle-like crystal of CBD2G. Then, the liquid is gently removed using a funnel with filter paper to collect the crystals which is quickly rinsed with a minimum volume of ice-cold distilled water. The collected white crystal is then dried in the vacuum oven for 24 hours to obtain CBD2G with >97% pure. The washed-water with the mother liquid may further be combined and filtered to further remove impurity (if any) followed by an additional 48 hours recrystallization to collect an additional ˜5 to 10% of CBD2G.

As used herein, the term “glycosylation,” or “glycosylating” refers to the coupling of a glycosyl donor, to a glycosyl acceptor, and preferably a cannabinoid forming a glycoside. Glycosylation of a glycosyl acceptor compound, such as a cannabinoid, is mediated by a UDP-glycosyltransferase (UGT) enzymes and may be accomplished in vitro or in vivo.

As used herein, the term “CBDIG,” means a CBD molecule having one UDP-sugar moiety. As used herein, the term “CBD2G,” means a CBD molecule having two UDP-sugar moieties. As used herein, the term “CBD3G,” means a CBD molecule having three UDP-sugar moieties.

As used herein, the term “solubility” is a measure of the maximum amount of solute that forms a homogeneous solution with a specified solvent under equilibrium conditions.

As used herein, the term “complex mixture,” or “mixture” means, a quantity of cannabinoids that may contain one or more one or more cannabinoids species, and one or more cannabinoid glycosides species For example, a solution, or lyophilized composition of a complex mixture may contain a CBD, a CBD1G species, a CBD2G species, and/or a CBD3G species, or a combination of the same. In another preferred embodiment, the term “complex mixture,” or “mixture” means, a quantity of two or more glycoside species each a different number of UDP-sugar moieties. For example, a solution, or lyophilized composition of a complex mixture of cannabinoid glycosides may contain a CBD1G species, a CBD2G species and CBD3G species, or a combination of the same.

As used herein, “Soxhlet” or “Soxhlet Solid-Phase Extraction device” means an apparatus or method generally having a configuration in which a flask containing a solvent at the bottom, a filter paper or a sintered glass containing a solid sample in the middle, and a cooling pipe at the top are provided. When the flask is heated, the solvent evaporates and condenses in the top cooling tube. When the solvent is dropped, the solvent drops on the solid sample, dissolves a small amount of the target component, and returns to the flask. In general, since the target component has a boiling point higher than that of the solvent, the target component is gradually concentrated in the flask by repeating this cycle. Since the refluxing solvent does not contain the target component, it does not saturate and can be extracted efficiently with a relatively small amount of solvent.

A “polar solvent” refers to a solvent which has a polarity, and which has a dielectric constant (∈) of 2.9 or greater, such as dimethylformamide (DMF), tetrahydrofuran (THF), MTHF, ethylene glycol dimethyl ether (DME), dimethylsulfoxide (DMSO), acetone, acetonitrile, methanol, ethanol, isopropanol, n-propanol, t-butanol, 2-methoxyethyl ether, ethyl acetate and isopropyl acetate.

A “non-polar solvent” refers to a solvent having no polarity or a solvent having relatively small polarity. Specific examples of the non-polar solvent according to the present invention can include pentane, diethyl ether, diisopropyl ether (isopropyl ether), hexane, heptane, tetrachloromethane, toluene, benzene, dichloromethane, chloroform, cyclohexane, and butyl acetate, and mixtures thereof with acetic acid ester, or methanol. As used herein, the terms “lipophilic properties,” or “lipophilicity” represents the affinity of a molecule for a lipidic environment. A compounds lipophilicity can be determined by measuring the partition coefficient, P, which is the ratio of solute concentrations in binary phases of organic and aqueous solvents, such as octanol and water, under equilibrium conditions.

As used herein, the term “sorption” refers to a process that results in the association of atoms or molecules with a target material. Sorption includes both adsorption and absorption. Absorption refers to a process in which atoms or molecules move into the bulk of a porous material, such as the absorption of water by a sponge. Adsorption refers to a process in which atoms or molecules move from a bulk phase (that is, solid, liquid, or gas) onto a solid or liquid surface. The term adsorption may be used in the context of solid surfaces in contact with liquids and gases. Molecules that have been adsorbed onto solid surfaces are referred to generically as adsorbates, and the surface to which they are adsorbed as the substrate or adsorbent. Adsorption is usually described through isotherms, that is, functions which connect the amount of adsorbate on the adsorbent, with its pressure (if gas) or concentration (if liquid). In general, “desorb,” “sorption,” and “desorption,” refers to the reverse of adsorption, and is a process in which molecules adsorbed on a surface are transferred back into a bulk phase.

As used herein, the terms “isolated” “pure” or “purified,” means that the in a heterogenous mixture containing both a cannabinoid or cannabinoid-glycoside, or a plurality of cannabinoid-glycoside species of the invention, one or more of the species has been substantially separated from the other species, such that, in ap referred embodiment only one, or a plurality of cannabinoid species comprises a majority, or the only species present, which may include greater than 50%, about 60%, about 70%, about 80%, about 90%, about 95%, about 98%, or about 99%. As used herein, a “cannabinoid” is a chemical compound (such as cannabinol, THC or cannabidiol) that is found in the plant species Cannabis among others like: Echinacea, Acmella Oleracea, Helichrysum Umbraculigerum; Radula marginata (Liverwort) and Theobroma cacao, and metabolites and synthetic analogues thereof that may or may not have psychoactive properties. Cannabinoids therefore include (without limitation) compounds (such as THC) that have high affinity for the cannabinoid receptor (for example Ki<250 nM), and compounds that do not have significant affinity for the cannabinoid receptor (such as cannabidiol, CBD). Cannabinoids also include compounds that have a characteristic dibenzopyran ring structure (of the type seen in THC) and cannabinoids which do not possess a pyran ring (such as cannabidiol). Hence a partial list of cannabinoids includes THC, CBD, dimethyl heptylpentyl cannabidiol (DMHP-CBD), 6,12-dihydro-6-hydroxy-cannabidiol (described in U.S. Pat. No. 5,227,537, incorporated by reference), (3S,4R)-7-hydroxy-Δ6-tetrahydrocannabinol homologs and derivatives described in U.S. Pat. No. 4,876,276, incorporated by reference; (+)-4-[4-DMH-2,6-diacetoxy-phenyl]-2-carboxy-6,6-dimethylbicyclo[3.1.1]hept-2-en, and other 4-phenylpinene derivatives disclosed in U.S. Pat. No. 5,434,295, which is incorporated by reference; and cannabidiol (−) (CBD) analogs such as (−) CBD-monomethylether, (−) CBD dimethyl ether; (−) CBD diacetate; (−) 3′-acetyl-CBD monoacetate; and ±AF11, all of which are disclosed in Consroe et al., J. Clin. Phannacol. 21: 428S-436S, 1981, which is also incorporated by reference. Many other cannabinoids are similarly disclosed in Agurell et al., Pharmacol. Rev. 38:31-43, 1986, which is also incorporated by reference.

The term “cannabinoid” may also include different modified forms of a cannabinoid such as a methylated, acetylated, hydroxylated cannabinoids or cannabinoid carboxylic acids. Examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol,trihydroxy-delta-9-tetrahydrocannabinol, and cannabinol.

A cannabinoid may include one or more conjugate sites or conjugation sites that can bind to a promoiety through a linker. As used herein “conjugate site” or “conjugation site” mean a position on a cannabinoid compound that may covalently bind to promoiety directly, or preferably through a linker that is coupled with promoiety, In one preferred embodiment, a “conjugate site” or “conjugation site” may include a OH or a COOH group on a cannabinoid.

As used herein the singular forms “a,” “an,” and “the” include plural referents unless the context clearly dictates otherwise. Thus, for example, reference to “a compound” includes a plurality of such compounds, and reference to “the method” includes reference to one or more methods, method steps, and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including,” as well as other related forms, such as “includes” and “included,” is not limiting. The term “about” as used herein is a flexible word with a meaning similar to “approximately” or “nearly.” The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

### TABLES

TABLE 1
Desorption of CBDIG using mixture of isopropanol
(IPA) and n-heptane (HEP) at 1:9 ratio (v/v)

| | Concentration (mg/mL) | | IPA:HEP | Solvent:resin | Desorp CBD1G | |
|---|---|---|---|---|---|---|
| Cycles | CBD1G | CBD | (mL) | ratio (mL/g) | Amount (g) | Percentage (%) |
| 1 | 4.085 | 1.136 | 135 | 6.4 | 0.552 | 29.71 |
| 2 | 2.634 | 0.444 | 130 | 6.2 | 0.342 | 18.45 |
| 3 | 1.986 | 0.179 | 130 | 6.2 | 0.258 | 13.91 |
| 4 | 1.616 | 0.095 | 128 | 6.1 | 0.207 | 11.14 |
| 5 | 0.844 | 0.028 | 130 | 6.2 | 0.110 | 5.91 |
| 6 | 0.695 | 0.016 | 128 | 6.1 | 0.089 | 4.79 |
| 7 | 0.520 | 0.011 | 130 | 6.2 | 0.068 | 3.64 |
| 8 | 0.386 | 0.009 | 130 | 6.2 | 0.050 | 2.70 |
| 9 | 0.398 | 0.008 | 130 | 6.2 | 0.052 | 2.79 |
| 10 | 0.214 | 0.007 | 125 | 6.0 | 0.027 | 1.44 |
| 11 | 0.187 | 0.009 | 120 | 5.7 | 0.022 | 1.21 |
| 12 | 0.123 | 0.013 | 125 | 6.0 | 0.015 | 0.83 |
| 13 | 0.098 | 0.010 | 132 | 6.3 | 0.013 | 0.70 |
| 14 | 0.052 | 0.008 | 125 | 6.0 | 0.007 | 0.35 |
| 15 | 0.041 | 0.008 | 140 | 6.7 | 0.006 | 0.31 |
| Total | | | 2008 | | 1.817 | 97.9 |

TABLE 2
Desorption of CBD2G using 2-methyl tetrahydrofuran (MTHF)

| | Concentration (mg/mL) | | MTHF | Solvent:resin | Desorp CBD2G | |
|---|---|---|---|---|---|---|
| Cycles | CBD2G | CBD1G | (mL) | ratio (mL/g) | Amount (g) | Percentage (%) |
| 1 | 5.736 | 0.340 | 66 | 3.1 | 0.379 | 24.42 |
| 2 | 7.354 | 0.237 | 66 | 3.1 | 0.485 | 31.31 |
| 3 | 4.152 | 0.000 | 65 | 3.1 | 0.270 | 17.41 |
| 4 | 2.351 | 0.000 | 62 | 3.0 | 0.146 | 9.40 |
| 5 | 1.350 | 0.000 | 70 | 3.3 | 0.095 | 6.10 |
| 6 | 0.664 | 0.018 | 66 | 3.1 | 0.044 | 2.83 |
| 7 | 0.395 | 0.000 | 60 | 2.9 | 0.024 | 1.53 |
| 8 | 0.225 | 0.000 | 65 | 3.1 | 0.015 | 0.94 |
| 9 | 0.134 | 0.000 | 65 | 3.1 | 0.009 | 0.56 |
| 10 | 0.128 | 0.000 | 65 | 3.1 | 0.008 | 0.54 |
| 11 | 0.064 | 0.000 | 66 | 3.1 | 0.004 | 0.27 |
| 12 | 0.036 | 0.000 | 65 | 3.1 | 0.002 | 0.15 |
| 13 | 0.020 | 0.000 | 65 | 3.1 | 0.001 | 0.09 |
| 14 | 0.000 | 0.000 | 66 | 3.1 | 0.000 | 0.00 |
| 15 | 0.000 | 0.000 | 65 | 3.1 | 0.000 | 0.00 |
| Total | | | 977 | | 1.481 | 95.6 |

TABLE 3
The amount of CBDs species absorb on the 21 gram oven dry resin

| Total amount loaded on the | | | Amount loaded per gram of dried | | |
|---|---|---|---|---|---|
| resin (g) | | | resin (g/g) | | |
| CBD2G | CBD1G | CBD | CBD2G | CBD1G | CBD |
| 1.5563 | 1.823 | 0.264 | 0.074 | 0.087 | 0.013 |

TABLE 4
Desorption of CBD and CBDIG using mixture of isopropanol (IPA) and n-heptane (HEP) at 1:9 ratio, v/v

| | | | Vol | | | | Amount desorb/remain per | |
|---|---|---|---|---|---|---|---|---|
| | | | IPA:HEP | Ratio of | | | gram of dried resin (g/g) | |
| | Concentration (mg/mL) | | (1:9, v/v) | sol:resin | Amount Desorp (g) | | Desorb | Remain |
| Cycles | CBD1G | CBD | (mL) | (mL/g) | CBD1G (g) | CBD (g) | (CBD1G) | (CBD1G) |
| Wet resin | | | 70 | 3.34 | 1.823 | 0.264 | 0.000 | 0.0870 |
| 1 | 4.09 | 1.14 | 135 | 6.44 | 0.552 | 0.153 | 0.026 | 0.061 |
| 2 | 2.63 | 0.44 | 130 | 6.20 | 0.342 | 0.058 | 0.016 | 0.044 |
| 3 | 1.99 | 0.18 | 130 | 6.20 | 0.258 | 0.023 | 0.012 | 0.032 |
| 4 | 1.62 | 0.10 | 128 | 6.11 | 0.207 | 0.012 | 0.010 | 0.022 |
| 5 | 0.84 | 0.03 | 130 | 6.20 | 0.110 | 0.004 | 0.005 | 0.017 |
| 6 | 0.69 | 0.02 | 128 | 6.11 | 0.089 | 0.002 | 0.004 | 0.013 |
| 7 | 0.52 | 0.01 | 130 | 6.20 | 0.068 | 0.001 | 0.003 | 0.009 |
| 8 | 0.39 | 0.01 | 130 | 6.20 | 0.050 | 0.001 | 0.002 | 0.007 |
| 9 | 0.40 | 0.01 | 130 | 6.20 | 0.052 | 0.001 | 0.002 | 0.005 |
| 10 | 0.21 | 0.01 | 125 | 5.96 | 0.027 | 0.001 | 0.001 | 0.003 |
| 11 | 0.19 | 0.01 | 120 | 5.73 | 0.022 | 0.001 | 0.001 | 0.002 |
| 12 | 0.12 | 0.01 | 125 | 5.96 | 0.015 | 0.002 | 0.001 | 0.002 |
| 13 | 0.10 | 0.01 | 132 | 6.30 | 0.013 | 0.001 | 0.001 | 0.001 |
| 14 | 0.05 | 0.01 | 125 | 5.96 | 0.007 | 0.001 | 0.000 | 0.001 |
| 15 | 0.04 | 0.01 | 140 | 6.68 | 0.006 | 0.001 | 0.000 | 0.000 |
| total | | | 2008 | | 1.817 | 0.263 | 0.0867 | |

| | |
|---|---|
| Total percentage CBD1G desorbed (%) | 99.7 |

TABLE 5
Desorption of CBD2G using water and 2-methyl tetrahydrofuran (MTHF)

| | | | | | | | Amount desorb/remain per | |
|---|---|---|---|---|---|---|---|---|
| | | | Vol | Ratio of | | | gram of dried resin (g/g) | |
| | Concentration (mg/mL) | | MTHF | sol:resin | Amount desorb (g) | | Desorb | Remain |
| Cycles | CBD2G | CBD1G | (mL) | (mL/g) | CBD2G (g) | CBD1G (g) | (CBD2G) | (CBD2G) |
| 0 | | | | | 1.555 | | | 0.0772 |
| 1 | 5.020 | 0.152 | 66 | 3.15 | 0.331 | 0.010 | 0.0158 | 0.0584 |
| 2 | 6.841 | 0.094 | 66 | 3.15 | 0.451 | 0.006 | 0.0215 | 0.0368 |
| 3 | 6.156 | 0.054 | 65 | 3.10 | 0.400 | 0.004 | 0.0191 | 0.0178 |
| 4 | 2.859 | 0.015 | 62 | 2.96 | 0.177 | 0.001 | 0.0085 | 0.0093 |
| 5 | 1.475 | 0.000 | 70 | 3.34 | 0.103 | 0.000 | 0.0049 | 0.0044 |
| 6 | 0.728 | 0.006 | 66 | 3.15 | 0.048 | 0.000 | 0.0023 | 0.0021 |
| 7 | 0.348 | 0.002 | 60 | 2.86 | 0.021 | 0.000 | 0.0010 | 0.0011 |
| 8 | 0.163 | 0.000 | 65 | 3.10 | 0.011 | 0.000 | 0.0005 | 0.0006 |
| 9 | 0.092 | 0.000 | 65 | 3.10 | 0.006 | 0.000 | 0.0003 | 0.0003 |
| 10 | 0.043 | 0.000 | 65 | 3.10 | 0.003 | 0.000 | 0.0001 | 0.0002 |
| 11 | 0.022 | 0.000 | 66 | 3.15 | 0.001 | 0.000 | 0.0001 | 0.0001 |
| 12 | 0.013 | 0.000 | 65 | 3.10 | 0.001 | 0.000 | 0.0000 | 0.0001 |
| 13 | 0.009 | 0.000 | 65 | 3.10 | 0.001 | 0.000 | 0.0000 | 0.0000 |
| 14 | 0.007 | 0.000 | 66 | 3.15 | 0.000 | 0.000 | 0.0000 | 0.0000 |
| 15 | 0.000 | 0.000 | 65 | 3.10 | 0.000 | 0.000 | 0.0000 | 0.0000 |
| total | | | 977 | | 1.555 | 0.001 | 0.0742 | |

| | |
|---|---|
| Total percentage CBD2G desorbed (%) | 96.1 |

### REFERENCES

1. Badiola I., Doshi A., Narouze S. Cannabis, cannabinoids, and cannabis-based medicines: Future research directions for analgesia. Reg. Anesth. Pain Med. 2022; 47:437-444.
2. Anand U., Pacchetti B., Anand P., Sodergren M. H. Cannabis-based medicines and pain: A review of potential synergistic and entourage effects. Pain Manag. 2021; 11:395-403.
3. Yenilmez F., Frundt O., Hidding U., Buhmann C. Cannabis in Parkinson's Disease: The Patients' View. J. Parkinson's Dis. 2021; 11:309-321.
4. Namdar D., Anis O., Poulin P., Koltai H. Chronological Review and Rational and Future Prospects of Cannabis-Based Drug Development. Molecules. 2020; 25:4821.
5. Ahuja A. S. Cannabis-based treatments as an alternative remedy for epilepsy. Integr. Med. Res. 2019; 8:200-201.
6. Grenier K., Ponnambalam F., Lee D., Lauwers R., Bhalerao S. Cannabis in the Treatment of Traumatic Brain Injury: A Primer for Clinicians. Can. J. Neurol. Sci. J. Can. Des Sci. Neurol. 2019; 47:11-17.
7. Fraguas-Sánchez A. I., Torres-Suárez A. I. Medical Use of Cannabinoids. Drugs. 2018; 78:1665-1703.
8. Aizpurua-Olaizola O, Soydaner U, Öztürk E, Schibano D, Simsir Y, Navarro P, et al. (February 2016). Evolution of the Cannabinoid and Terpene Content during the Growth of Cannabis sativa Plants from Different Chemotypes. Journal of Natural Products. 79 (2): 324-331.
9. Sayre R. T., Goncalves E. C., Zidenga T. High Level in Vivo Biosynthesis and Isolation of Water Soluble Cannabinoids in Stably Transformed Plant Systems. 0338301A1. U.S. Patent. 2019
10. Sayre R. T., Goncalves E. C., Zidenga T. Generation of Water-Soluble Cannabinoid Compounds in Yeast and Plant Cell Suspension Cultures and Compositions of Matter. 078168A1. U.S. Patent. 2019

1. A method for the extraction of cannabidiol (CBD) glycoside species from a complex mixture comprising:
contacting a crude extract containing a quantity of CBD and CBD-glycoside with a polar solvent or non-polar solvent;
isolating the solvent containing the CBD-glycosides and further washing it with a quantity of a base and a salt forming an organic layer containing the concentrated CBD-glycosides; and
isolating the CBD-glycoside from said organic layer.

2. The method of claim 1, wherein said crude extract comprises a fermented crude extract.

3. The method of claim 1, wherein said CBD-glycoside comprises a complex mixture containing CBD-glycosides having between one UDP-sugar moiety (CBD1G), two UDP-sugar moieties (CBD2G), or three UDP-sugar moieties (CBD3G), or four UDP-sugar moieties (CBD4G).

4. The method of claim 1, wherein said polar solvent is selected from: 2-methyl tetrahydrofuran (MTHF), ethyl acetate, or isopropyl acetate, or a combination of the same.

5. The method of claim 1, wherein said base comprises sodium bicarbonate.

6. The method of claim 1, wherein said salt comprises sodium chloride.

7. The method of claim 1, wherein the CBD-glycoside comprises CBD1G, CBD2G, or a combination of the same.

8. The method of claim 1, wherein the isolated CBD-glycoside comprises solid CBD1G, solid CBD2G, or a combination of the same

9. The method of claim 8, further comprising the step of purifying said solid CBD1G and/or CBD2G.

10. The method of claim 9, wherein said step of purifying comprises contacting said solid CBD1G and/or CBD2G with a quantity of a polar solvent and filtering the resulting solution and allowing said CBD1G and/or CBD2G to undergo primary crystallization.

11. The method of claim 10, wherein said polar solvent is selected from: acetone, or propanol.

12. The method of claim 10, further comprising contacting an uncrystallized portion of the solution with a non-polar solvent and allowing the remaining uncrystallized CBD1G and/or CBD2G to undergo primary crystallization.

13. The method of claim 12, wherein said non-polar solvent is selected from: heptane, or hexane.

14. The method of claim 10, wherein the primary crystallized CBD1G and/or CBD2G is contacted with solution containing deionized water and a polar solvent for secondary crystallization of the CBD1G and/or CBD2G.

15. The method of claim 14, wherein the polar solvent for secondary crystallization is selected from: acetone, ethanol, and methanol.

16. The method of claim 10, wherein the step of precipitation and/or crystallization occur at room temperature

17. The method of claim 9, wherein said step of purifying comprises the step of contacting the CBD1G and/or CBD2G with a quantity of methanol or other polar solvent and filtering the resulting solution and allowing the CBD1G and/or CBD2G to crystallize.

18. A method for the extraction of cannabinoid glycoside species from a complex mixture comprising:
contacting a crude extract containing a quantity of a cannabinoid and a cannabinoid glycoside with a polar solvent, wherein the polarity of the solvent causes the cannabinoid glycoside to be separated from the extract;
isolating the solvent containing the cannabinoid glycoside and further washing it with a quantity of a base and a salt forming an organic layer containing the concentrated cannabinoid glycoside; and
isolating the cannabinoid glycoside from said organic layer.

19. The method of claim 18, wherein said crude extract comprises a fermented crude extract.

20. The method of claim 18, wherein said cannabinoid glycoside comprises a complex mixture containing one or more CBD-glycosides having between one UDP-sugar moiety (CBD1G), two UDP-sugar moieties (CBD2G), three UDP-sugar moieties (CBD3G), or four UDP-sugar moieties (CBD4G).

21. The method of claim 18, wherein said polar solvent is selected from: 2-methyl tetrahydrofuran (MTHF), ethyl acetate or isopropyl acetate.

22. The method of claim 18, wherein said base comprises sodium bicarbonate.

23. The method of claim 18, wherein said salt comprises sodium chloride.

24. The method of claim 18, wherein the cannabinoid comprises CBD and said cannabinoid-glycoside comprises CBD1G, CBD2G, or a combination of the same.

25. The method of claim 18, wherein the isolated CBD-glycoside comprises solid CBD1G, solid CBD2G, or a combination of the same

26. The method of claim 25, further comprising the step of purifying said solid CBD1G and/or CBD2G.

27. The method of claim 26, wherein the step of purifying comprises contacting said solid CBD1G and/or CBD2G with a quantity of a polar solvent and filtering the resulting solution and allowing the CBD1G and/or CBD2G to undergo primary crystallization.

28. The method of claim 27, wherein said polar solvent is selected from: acetone, or propanol.

29. The method of claim 27, further comprising contacting an uncrystallized portion of the solution with a non-polar solvent and allowing the remaining uncrystallized CBD1G and/or CBD2G to undergo primary crystallization.

30. The method of claim 29, wherein said non-polar solvent is selected from: heptane, or hexane.

31. The method of claim 27, wherein the primary crystalized CBD1G and/or CBD2G undergoes secondary crystallization wherein the primary crystallized CBD1G and/or CBD2G is contacted with solution containing deionized water and a polar solvent for secondary crystallization.

32. The method of claim 31, wherein the polar solvent for secondary crystallization is selected from: acetone, ethanol, and methanol.

33. The method of claim 29, wherein the step of precipitation and/or crystallization occur at room temperature.

34. The method of claim 26, wherein said step of purifying comprises the step of contacting the CBD1G and/or CBD2G with a quantity of methanol or other polar solvent filtering the resulting solution and allowing said CBD2G to crystallize.

35-129. (canceled)
